(19) United States
(12) Patent Application Publication
(10) Pub. No.: US 2016/0055303 A1
(43) Pub. Date: Feb. 25, 2016

(54) DEVICE AND METHOD FOR SUPPORTING THE OPERATION OF MEDICAL EQUIPMENT

(71) Applicant: FRESENIUS MEDICAL CARE DEUTSCHLAND GMBH, Bad Homburg (DE)

(72) Inventor: Burkard KELLER, Wuerzburg (DE)

(21) Appl. No.: 14/779,461

(22) PCT Filed: Apr. 4, 2014
(86) PCT No.: PCT/EP2014/056862

§ 371 Date: Sep. 23, 2015

(30) Foreign Application Priority Data
Apr. 5, 2013 (DE) 10 2013 005 743.4

(51) Int. Cl. G06F 19/00; A61M 1/34

(52) U.S. Cl. CPC G06F 19/3406; A61M 1/3403; A61M 1/3496

(57) ABSTRACT

The present invention relates to the field of medical machines. The invention is based on the object of facilitating the operation of medical machines and making them safer. To do so, the invention proposes methods and devices with which a transparent display can be applied in front of at least parts of medical machines so that these parts of the medical machines are visible through the transparent display and information such as operating instructions or messages are displayed on the transparent display.

The present invention relates to the field of medical equipment, in particular blood treatment machines such as dialysis machines and the operation of same.

### BACKGROUND

Blood treatment machines are medical machines in which blood from a patient is treated, usually in an extracorporeal circulation. For example, drugs may be added to the blood, or constituents of the blood may be removed from it, or the blood may be heated or cooled. These processes are often monitored by sensors. For these purposes, blood treatment machines have a number of devices such as pumps, valves, sensors, clamps or the like. In addition, disposable medical articles are often used for blood treatment. Such disposable articles are usually discarded after the treatment for reasons of hygiene. Disposable medical articles or so-called disposables include, for example, tubing sets for carrying medical fluids such as blood or drugs, filters such as adsorber filters or dialysis filters, bags for storing drugs and the like. These disposables are usually inserted into the blood treatment machine by the medical personnel and/or are connected to the blood treatment machine and then removed or separated from the blood treatment machine after the treatment is completed. In this context, we also speak of setting up and stripping down the blood treatment machine.

The devices that must be operated by the medical personnel are often arranged on a central surface or level of the blood treatment machine, which is easily reached and easily observed, in order to facilitate operation and to improve the overview. Such an area is called an operating zone in accordance with the teaching of the present invention.

The present invention will now be explained in greater detail below on the basis of the example of the dialysis machine as one embodiment of a medical machine.

Dialysis machines are blood treatment machines, in which a fluid from a patient is sent through a fluid line to a fluid treatment component, where it is treated by the fluid treatment component and then is returned to the patient through the fluid line, which may be divided into an arterial branch and a venous branch. Examples of such blood treatment machines include hemodialysis machines in particular. One such blood treatment machine is the subject matter of DE 198 49 787 C1 by the present applicant, the contents of which are herewith fully included in the disclosure content of the present patent application.

Dialysis is a method for purifying the blood of patients with acute or chronic renal failure. A fundamental distinction is made here between methods using an extracorporeal blood circulation, such as hemodialysis, hemofiltration or hemodiafiltration and peritoneal dialysis, which does not have an extracorporeal blood circulation.

In hemodialysis in an extracorporeal circulation, blood is passed through the blood chamber of a dialyzer, which is separated by a semipermeable membrane from a dialysis fluid chamber. A dialysis fluid that contains the blood electrolytes in a certain concentration flows through the dialysis fluid chamber. The concentration of blood electrolytes in the dialysis fluid corresponds to the concentration in the blood of a healthy person. During the treatment, the patient's blood and the dialysis fluid are usually passed by both sides of the semipermeable membrane in counter- current at a predetermined flow rate. The substances that must be eliminated in the urine diffuse from the blood chamber through the membrane and into the chamber for the dialysis fluid, while at the same time electrolytes that are present in the blood and in the dialysis fluid diffuse from the chamber having the higher concentration to the chamber having the lower concentration. If a pressure gradient is built up on the dialysis membrane from the blood side to the dialysate side, for example, by means of a pump, which withdraws dialysate from the dialysate circulation downstream from the dialysis filter on the dialysate side, water goes from the patient's blood through the dialysis membrane and into the dialysate circuit. This process of ultrafiltration leads to the desired withdrawal of water from the patient's blood.

In hemofiltration, ultrafiltrate is withdrawn from the patient's blood by applying a transmembrane pressure in the dialyzer without passing dialysis fluid by the side of the membrane of the dialyzer opposite the patient's blood. In addition, a sterile and pyrogen-free substituate solution may be added to the patient's blood. This is called predilution or postdilution, depending on whether the substituate solution is added upstream or downstream from the dialyzer. The mass exchange takes place by convection in hemofiltration.

Hemodiafiltration combines the methods of hemodialysis and hemofiltration. A diffusive mass exchange takes place between the patient's blood and the dialysis fluid through the semipermeable membrane of a dialyzer and plasma water is also filtered through a pressure gradient on the membrane of the dialyzer.

The methods of hemodialysis, hemofiltration and hemodiafiltration are usually performed using automotive hemodialysis machines such as those distributed by the applicant under the designation 5008, for example.

Plasmapheresis is a blood treatment method in which the patient's blood is separated into blood plasma and its corpuscular constituents (cells). The separated blood plasma is purified or replaced by a replacement solution, i.e., substituate, and the purified blood plasma or replacement solution is returned to the patient.

In peritoneal dialysis, a catheter is passed through the abdominal wall to fill the patient's abdominal cavity with a dialysis fluid having a concentration gradient with respect to the endogenous fluids. The toxins present in the patient's body migrate through the peritoneum, which acts as a membrane, and then they enter the abdominal cavity. After a few hours, the dialysis fluid, which is now spent fluid, in the patient's abdominal cavity is replaced. Water can be transferred from a patient's blood through the peritoneum and into the dialysis fluid based on osmotic processes and thus withdraws water from the patient.

The peritoneal dialysis method is usually performed with the help of automatic peritoneal dialysis systems, such as those distributed by the applicant under the brand name sleep.safe.

Dialysis machines, as an example of complex medical fluid management machines, have extensive functions. To control these functions, medical fluid management machines such as dialysis machines are equipped with at least one control unit, which may be embodied as a CPU (central processing unit) or as a software-programmable microcontroller. Such devices are often operated by touchscreen displays. Such a touchscreen display combines an input and output device in one surface, in that it provides a touch-sensitive surface with which user input can be detected.

Information such as operating instructions or messages pertaining to certain parts of the dialysis machine can be output on such a display device. This may take place in particular in upgrading the machine with accessory parts before treatment and when these accessory parts are removed after a treatment.

Text instructions may be displayed here to indicate the instructions, optionally replaced by images, in particular pictograms representing the respective action or parts of the dialysis machine on which certain actions are to be performed.

Text messages and/or images directed at and/or representing certain parts of the dialysis machine may also be displayed during the treatment, for example, in the case of a malfunction of certain parts, which can be detected by corresponding sensors.

Since the display device and the respective parts of the dialysis machine are arranged at separate locations from one another, the operator must first locate the respective part of the dialysis machine when necessary. This may take up valuable time, in particular with an inexperienced operator or in the case of unclear diagrams on the display device, or it may lead to mistakes in operation in that case.

### DESCRIPTION

The object of the present invention was therefore to create a machine and a method which would improve the display of information such as operating instructions or messages and would prevent mistakes in operation and/or would facilitate the operation of technical medical equipment.

These objects are achieved according to the present invention by the method having the features of claim 1, a device having the features of claim 10, a computer program having the features of claim 18 and a computer program product having the features of claim 19.

Advantageous embodiments of the subject matter of the dependent claims.

Transparent displays are known from the state of the art. Such a display is transparent, which means that an object behind the display in the direction of viewing is discernible by the observer through the display.

Any graphics or texts can be shown on the display itself, so that an observer sees the displayed graphics or text in front of the transparent background.

U.S. Pat. No. 7,413,233, for example, discloses a foldable transparent LC display (liquid crystal display), which an automobile driver can see through but which is at the same time capable of displaying information such as navigation instructions.

The present invention refines this principle and applies it to technical medical equipment.

According to one embodiment of the invention, instead of a transparent LCD, an OLED (organic light-emitting diode) display is to be used. OLED displays known from the state of the art are mainly known as display devices of small portable devices, such as mobile telephones or digital cameras. Such displays are self-illuminating displays in contrast with LCD displays.

Like a silicon-based light-emitting diode, an organic light-emitting diode is made of two layers comprising a p-doped semiconductor material and an n-doped semiconductor material. A pn-junction is formed at the contact surface of these two semiconductor layers, causing energy to be released in the form of light radiation as a result of current flowing through the pn-junction in recombination of electrons with atoms missing an electron (so-called holes).

In organic light-emitting diodes, the semiconductor layer consists of organic semiconductors such as carbon compounds.

In a special type of embodiment, the semiconductor layers in organic light-emitting diodes may consist of transparent layers. A display comprised of a plurality of such individual OLEDs, which are controlled individually, is thus transparent.

Another advantage of OLED displays in comparison with conventional LCD displays is the possibility of applying the light-generating semiconductor lasers of OLED displays to flexible or curved surfaces. Thus, there are known OLED displays, which have a film carrier and are therefore flexible. US 2010 259 166A discloses such an OLED display applied to a flexible substrate.

Another advantage of OLED displays in comparison with conventional displays consists of a simple manufacturing process in which the semiconductor layers of the OLED display are applied to flexible supports in a spray process.

For example, WO06041240 describes a method with which OLED structures can be applied to a substrate which may be a flexible film, by using a type of inkjet printing technique.

It is also conceivable that such display structures are applied directly to a suitable surface. Thus, in light of the present invention, it is conceivable that a transparent plastic part can function as a carrier for a display applied to this plastic part, by spraying, for example.

According to one embodiment of the invention, transparent displays, regardless of the technology by which they operate or by which they are manufactured are applied in front of certain parts of a medical device, so that an observer can recognize these parts through the transparent display, and additionally, any desired graphics or texts can be shown on the display.

In one embodiment of the invention, a transparent display is applied in front of the operating zone of the medical machine embodied as a dialysis machine.

Messages in the form of graphs or texts may be shown on the transparent display and may be linked to the parts which are situated behind the location on which the corresponding message is displayed as seen in the direction of viewing.

For example, if there is a need to perform actions on a pump on a medical device or to direct the attention of the user to this pump, a message may be displayed at this location on the transparent display in front of this pump.

This message may include a text message. The message may also include a graphic. It is provided that the graphic is an image of the part of the medical machine behind the display screen. An area that is provided with a color or a pattern and is provided with most of the area in front of the part that is linked to the message to be displayed is provided. A distinction is made here between the color or the pattern and the rest of the display to permit a differentiation and to direct the user's attention to the parts of the medical machine mounted behind this surface.

It is also provided that the complete image of the surface, which is located on the medical machine behind the display, is itself displayed on the transparent display. Likewise, alternatively or additionally, any texts or graphics may also be displayed, for example, measured values or instrument settings as examples of operating parameters of the medical machine. Additional information which can be displayed in this way may include operating instructions for the medical machine or messages pertaining to the medical machine or relating to a treatment performed with the medical machine.

Another aspect of the present invention relates to the combination of a transparent display with a device which cancels or reduces this transparency in a controllable manner. Such a device for reducing transparency may be, for example, a liquid crystal cell, which changes its optical condition from transparent to opaque when a voltage is applied. US 200910015740 A1 describes such a system for darkening a glass window.

When such a system is applied to the rear side of a transparent display, the degree of transparency can be altered from completely transparent to completely opaque by corresponding control of one or more liquid crystal cells. The transparency can also be controlled locally by using several liquid crystal cells distributed spatially in the manner described above.

### BRIEF DESCRIPTION OF FIGURES

Additional details and advantages of the invention are described in greater detail below on the basis of exemplary embodiments as depicted in the drawings, in which:

FIG. 1 shows a conventional medical machine, embodied here as a dialysis machine having a conventional display;

FIG. 2 shows a conventional medical machine, embodied here as a dialysis machine having a two-piece transparent and foldable cover;

FIG. 3 shows a medical machine, for example, as a dialysis machine in accordance with the teaching of the present invention with a display applied to a two-piece foldable and transparent cover;

FIG. 4 shows the medical machine according to FIG. 3, with a partially open transparent cover, to which a display is applied in accordance with the teaching of the present invention;

FIG. 5 shows a detailed diagram of the display from FIG. 3 with an example of display contents in accordance with the teaching of the present invention;

FIG. 6 shows an alternative detailed diagram of the display from FIG. 3 with an example of display contents in accordance with the teaching of the present invention, and

FIG. 7 shows a schematic diagram of the components of a medical machine in accordance with the teaching of the present invention.

### DETAILED DESCRIPTION OF THE FIGURES

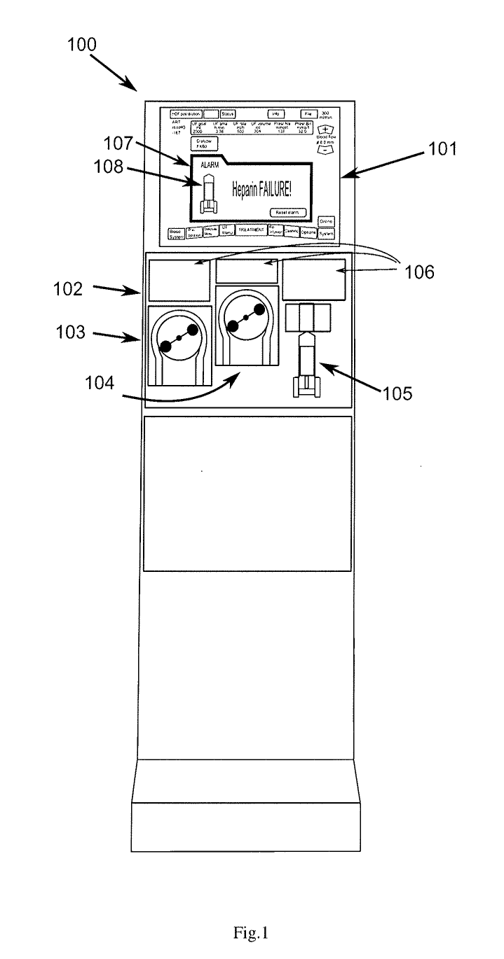

FIG. 1 shows a conventional medical machine, embodied here as a hemodialysis machine 100, in a simplified diagram. The hemodialysis machine 100 has a display 101 which may be a touchscreen display. In addition, the hemodialysis machine 100 comprises various parts which can be operated by the operator of the machine or which cooperate with other parts such as blood tubing or the like.

These parts are arranged in a field 102 in FIG. 1 and comprise, for example, two medical pumps 103 and 104, which are designed as hose roller pumps. The field 102 thus forms an operating zone in the sense of the invention. Such pumps are often used for pumping a patient's blood or the substituate fluid in hemodialysis treatments. In addition, the fields 106 symbolize, for example, units, sensors or operating fields having keys, displays or switches that are not shown in detail here.

In addition, FIG. 1 shows an injection pump 105, which is frequently used for pumping an anticoagulant such as heparin in hemodialysis treatments.

An anticoagulant which is added to the patient's blood in the extracorporeal blood circulation should prevent the patient's blood from coagulating in the extracorporeal blood circulation and clogging the dialysis filter, for example, or even being returned to the patient's vascular system, which can lead to serious health problems.

It is thus especially important for the hemodialysis treatment that a certain dose of an anticoagulant is supplied constantly and reliably to the patient's blood. Because of the low delivery rates, injection pumps have become established for this purpose. Proper functioning of these pumps can be checked by a variety of means, and an alarm message may be output in the event of a malfunction, so that the operating personnel can take appropriate measures as soon as possible.

FIG. 1 shows one such alarm message in a bordered field 107 of the display 101. In addition to the text message, a symbolic diagram 108 of the heparin pump 105 is also displayed in the field 107 to increase the effect of the alarm message on the operating personnel. In addition, the symbolic diagram of the heparin pump also provides information to the operator about how the heparin pump will appear to the operator in the specific case in order to prevent mixups. Furthermore, the diagram of the part associated with an alarm message is also recognizable from a distance, whereas the text messages are legible only from a closer distance under some circumstances.

Nevertheless, the alarm message and/or the diagram of the part of the dialysis machine to which the alarm message pertains, i.e., the heparin pump in FIG. 1, is separated from this part and is displayed at a distance.

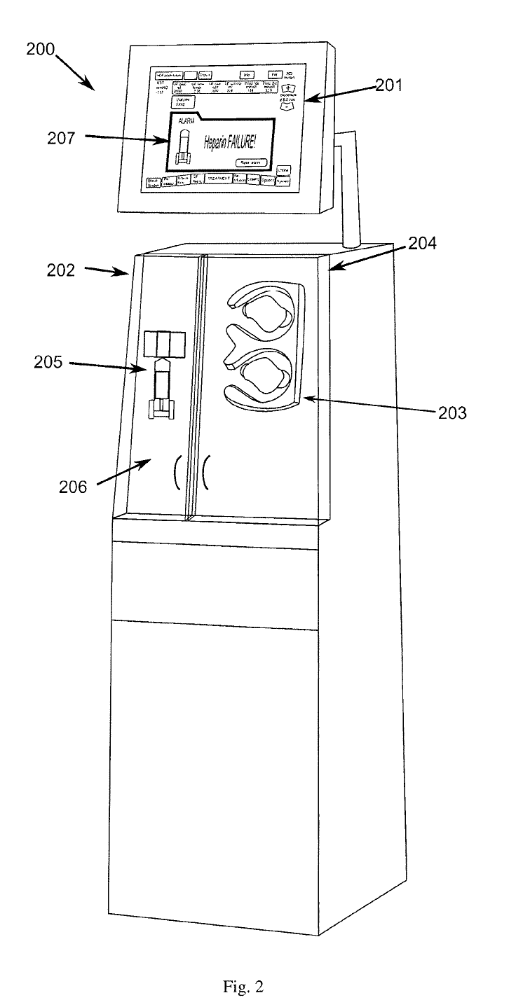

FIG. 2 shows another embodiment of a medical device embodied as a hemodialysis machine 200, for example. This hemodialysis machine also includes a display 201, which may be a touchscreen display.

As in FIG. 1, here again, heparin pump 205 and two hose roller pumps which are combined under reference numeral 203, are shown in FIG. 1.

In contrast with the hemodialysis machine 100 from FIG. 1, the hemodialysis machine 200 comprises transparent covers 202 and 204, which are embodied here as rotatably mounted doors. These doors 202 and 204 cover the units, which are on the surface 206 in the closed state, i.e., sensors, operating fields with keys, displays or switches. The surface 206 forms an operating zone in the sense of the present invention. Such a cover may be connected rotatably, tiltably and/or pivotably to the medical machine via a corresponding mechanical suspension such as hinges or articulated joints.

Recesses (not shown in FIG. 1) for the passage of blood tubings in the doors 202 and 204, for example, ensure that the doors can be closed tightly during the dialysis treatment.

Therefore this yields a uniform smooth surface and also noises that may originate from units such as pumps are dampened by the closed doors.

As in FIG. 1 the display 201 also shows an alarm message in a field 207, this alarm message being linked here to the heparin pump 205 as an example.

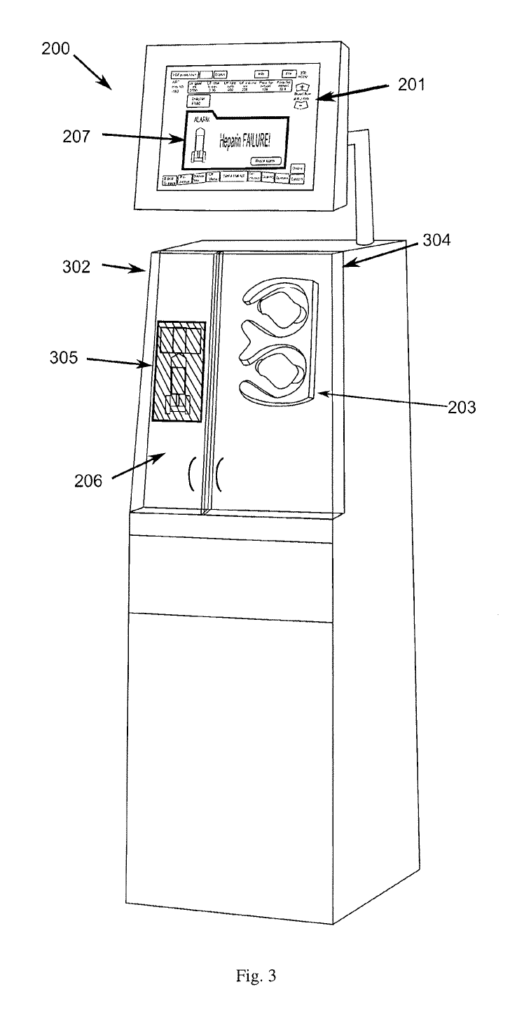

In accordance with the teaching of the present invention, FIG. 3 shows a medical machine like the one shown in FIG. 2 where the doors 302 and 304 are each designed as a transparent display and/or transparent displays are integrated into each. These two displays are mounted in front of the operating zone 206.

In contrast with FIG. 2, the alarm message which is associated with the heparin pump is shown in FIG. 3 not only on the display 201 but also instructions indicating where the heparin pump is located, namely behind the hatched area 305 in the direction of viewing, are also given on the transparent display, which is integrated into the door 302.

The hatched area 305 is displayed by the transparent display integrated into the door 302 in accordance with the alarm message. The display as a hatched area makes FIG. 3 more understandable, any graphical emphasis being possible to illustrate the location of the respective component.

In accordance with the teaching of the present invention, any graphics or texts can be displayed at any locations.

In accordance with the teaching of the present invention, these graphics or texts may also be displayed in animated form, for example, as flashing lights. In general, all displays that are possible with a conventional display are also conceivable here in accordance with the teaching of the present invention. It is essential that the display must unambiguously indicate a portion of the surface behind the transparent display in the direction of viewing.

This may be accomplished as shown in FIG. 3, for example, by displaying an area of any desired shape in front of the respective part. This area may have any desired design and may also comprise colors, patterns or animations in any combination as features suitable for visually emphasizing the surface as distinct from the remaining area and drawing the attention of the observer to the part located behind this surface.

In accordance with the teaching of the present invention, a precise image of the respective part may also be displayed for this purpose. The image may be displayed directly in front of the part being imaged.

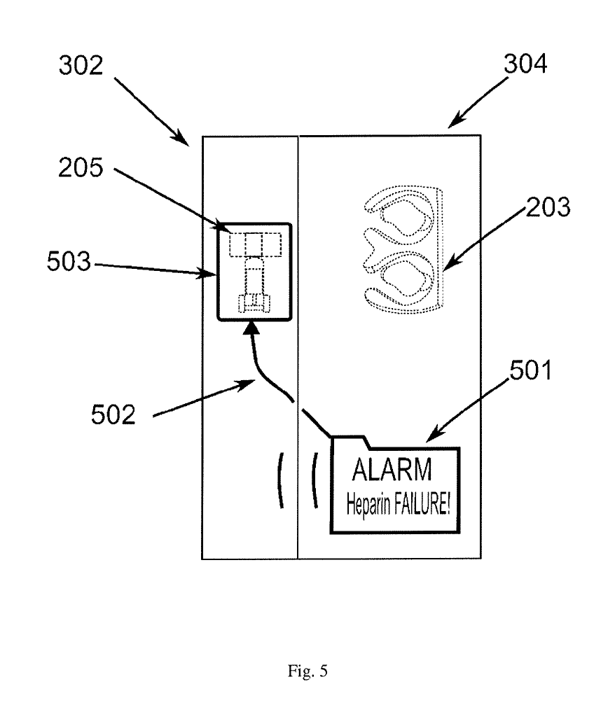

Alternatively, the display may also be located somewhere else, for example, at a location behind which there is a flat surface for better visibility. In this case, the display may be supplemented by additional information about the location of the part being displayed. Such information may be an arrow, for example, which can be seen pointing from this diagram to the corresponding part as shown in FIG. 5.

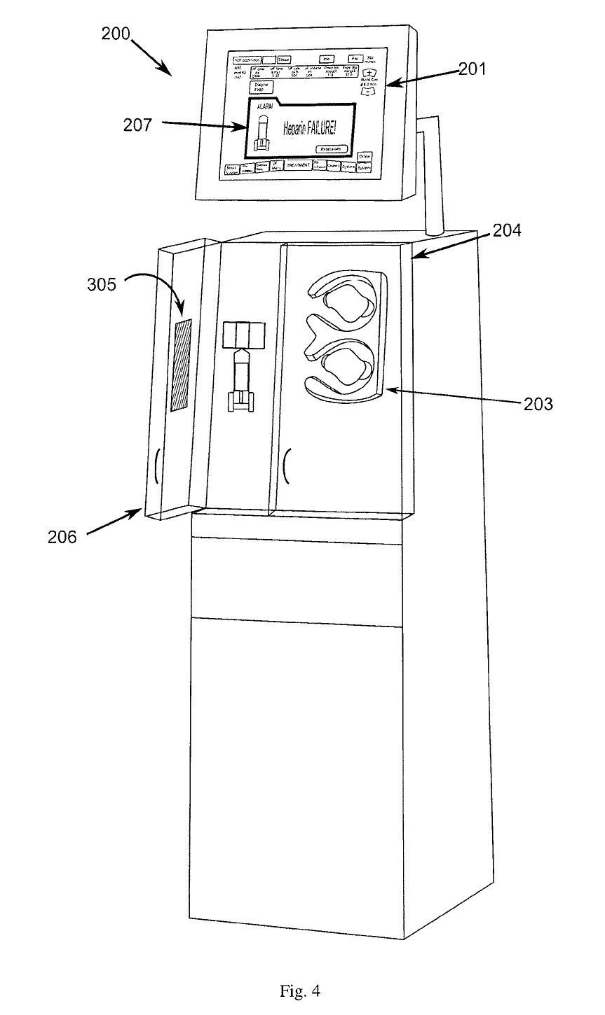

FIG. 4 shows the dialysis machine to illustrate the invention with an open door 302 which is embodied as a transparent display and on which an area 305 is shown with hatching in a situation like that shown in FIG. 3.

FIG. 5 shows an alternative display of the doors 302 and 304, which are embodied as transparent displays. The pumps 203 and 205 are shown with hatching to illustrate that they are situated behind the doors 302 and 304 and are visible through them for an observer.

FIG. 5 shows the display of an alarm message 501 in a readily visible location of the door 304 which is designed as a transparent display and behind which there is a smooth surface in this example, which facilitates the readability of the display as a background for the transparent display.

In addition to the warning message, there is a reference to the location of the heparin pump to which the alarm message 501 is directed by the arrow 502 in FIG. 5 and the rectangular frame 503, which form a type of graphic link.

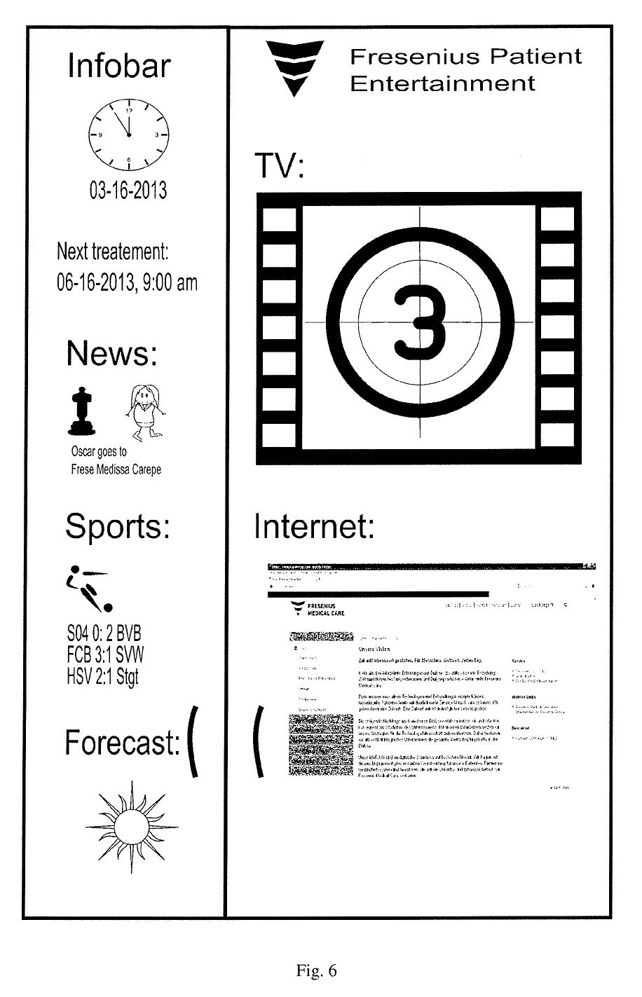

FIG. 6 shows an example of a display on an arrangement in accordance with the present invention, in which a transparent display is mounted in front of a device having one or more controllable liquid crystal cells in order to prevent or even eliminate the transparency of the display by controlling one or more liquid crystal cells with an electronic control signal. This may optionally also be accomplished locally in different ways over the area of the display, so that many areas are transparent but others are opaque.

Such an embodiment with the combination of two display technologies is advantageously used to display patient information with which a transparency of the display would lead to inferior recognizability,

Thus FIG. 6 shows examples of information directed at the patient. Transparent parts of the dialysis machine would interfere with the display, so in this example, the transparency is cancelled by a suitably controlled device having one or more controllable liquid crystal cells.

If OLED technology can be used for the transparent display in a preferred embodiment, then a brilliant image can be created even with a completely black opaque background display, because OLED displays are self-illuminating displays and thus do not require any background brightness for displaying the content of images.

In another embodiment of the present invention, the transparency of the display can be preserved at certain locations but prevented at other locations. This is advantageous in particular when parts of the medical machine which require special attention are located behind the transparent areas.

It is fundamentally conceivable that the displays described here may be equipped with a touch-sensitive layer. Then they may be used as a high-value substitute for conventional touchscreen displays.

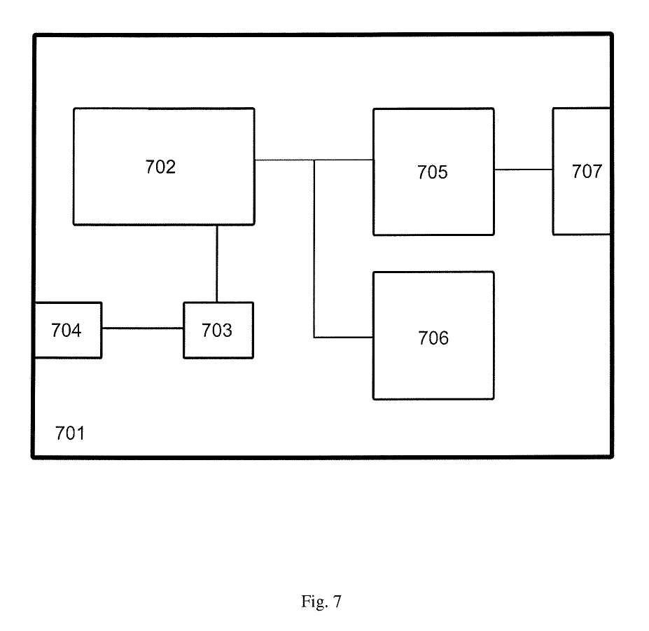

FIG. 7 shows a schematic diagram of the components of a medical machine 701 in agreement with the teaching of the present invention.

Such medical machines have a control unit 702, which is often embodied as a microprocessor. The control unit is programmed with software. To this end, the control unit has a data and program memory 703 as an internal memory, in which data and computer programs can be stored.

The computer programs may be saved on data media as computer program products comprising computer-readable program means. The data media can be used in a computer and also comprise storage in networks such as the Internet to which the user can have access, in addition to physical memories, such as diskettes, CD-ROMs, memory cards, USB sticks or DVDs. The computer programs comprise software code sections with which the methods described here can be executed when they are running on the control unit. To this end, the computer programs are loaded into the internal memory.

The data and program memory may be written and read out via any interface 704, for example.

In addition, the medical machine has at least one display 705, which is designed as a transparent display. In one embodiment, the medical machine has a display and/or a device for preventing transparency 706 which is arranged behind the respective transparent display in the direction of viewing.

In one embodiment the transparent display 705 may also have an input device 707 with a touch-sensitive layer in the sense of a touchscreen.

The control unit 702 is programmed by a suitably programmed computer program stored in the data and program memory 703, such that it controls at least one transparent display 705 and optionally the display and/or a device for preventing transparency 706 in the manner already described.

1. A method for supporting the operation of medical equipment, comprising the step:
Display of information on a transparent display, such that the transparent display is arranged at least partially in front of at least one part of the medical machine as seen in the direction of viewing.

2. The method according to claim 1, wherein the information includes a graphical emphasis, which indicates a portion of the medical machine.

3. The method according to claim 1, wherein the graphical emphasis is displayed in front of the part as seen in the direction of viewing.

4. The method according to claim 1, wherein a graphical linkin addition to the part of the medical machine is displayed or indicated by the graphical emphasis.

5. The method according to claim 2, wherein the or emphasis comprises a diagram of part of the medical, machine.

6. The method according to claim 1, wherein the graphical emphasis is displayed with animation.

7. The method according to claim 1, wherein the information comprises operating parameters of the medical machine, operating instructions for the medical machine, messages pertaining to the medical machine and/or it relates to a treatment performed with the medical machine.

8. The method according to claim 1, wherein the transparent display is combined with a device for reducing transparency, and the transparency of the combination of transparent display with the device for reducing transparency is controlled in such a way that the transparency of the combination is reduced in a spatially resolved manner.

9. The method according to claim 8, wherein the device for reducing transparency comprises at least one liquid crystal cell whose transparency is altered by applying an electric control signal.

10. The method according to claim 1, wherein the part is an operating zone of the medical machine.

11. A device comprised of a medical machine having a control unit and a transparent display, wherein the transparent display is arranged at least partially in front of a part of the medical machine, as seen in the direction of viewing.

12. The device according to claim 11, wherein the transparent display consists of individual transparent organic light-emitting diodes (OLEDs).

13. The device according to claim 11, additionally comprising a device for reducing transparency, which is arranged in front of or behind the transparent display, such that the transparency of the arrangement is variable by controlling the device to reduce transparency.

14. The device according to claim 13, wherein the device for reducing transparency comprises a least one liquid crystal cell whose transparency is variable by applying an electric control signal.

15. The device according to claim 14, wherein the transparent display and/or the arrangement of the transparent. display and the device for reducing transparency is embodied as a rotatable, tiltable and/or pivotable cover of the medical machine.

16. The device according to claim 11, wherein the control unit is configured to display information on the transparent display and/or to reduce the transparency of the arrangement of transparent display and the device for reducing transparency and this is done by controlling the device for reducing transparency, such that the reduction in transparency may be implemented in a spatially resolved manner.

17. The device according to claim 11, wherein the medical machine is a blood treatment machine.

18. The device according to claim 17, wherein the blood treatment machine is a dialysis machine and is equipped in particular for hemodialysis, for hemofiltration, for hemodiafiltration, for plasmapheresis or for automatic peritoneal dialysis.

19. The device according to claim 11, additionally comprising a touch-sensitive layer which together with the transparent display forms a touchscreen display.

20. The device according to claim 11, wherein the part is an operating zone of the medical machine.

21. A computer program, which can be loaded into the internal memory of at least one control unit and comprises software code sections with which the steps of the method according to claim 1 are executed when the computer program runs on the at least one control unit.

22. A computer program product saved on a data medium. that may he used in a computer, comprising computer-readable program means with which a computer can execute the method according to claim 1.
